(19) United States
(12) Patent Application Publication
(10) Pub. No.: US 2004/0197399 A1
(43) Pub. Date: Oct. 7, 2004

(54) N-fatty acid-amino acid conjugates and therapeutic uses

(75) Inventors: Sumner H. Burstein, Framingham, MA (US); Robert B. Zurier, Princeton, MA (US)

Correspondence Address: FISH & RICHARDSON PC, 225 FRANKLIN ST, BOSTON, MA, 02110, US

(73) Assignee: University of Massachusetts, a Massachusetts corporation

(21) Appl. No.: 10/747,030

(22) Filed: Dec. 23, 2003

Related U.S. Application Data
(63) Continuation of application No. 10/180,780, filed on Jun. 25, 2002, now abandoned.
(60) Provisional application No. 60/300,683, filed on Jun. 25, 2001.

(51) Int. Cl. A61K 31/198; A61K 9/20

(52) U.S. Cl. 424/464; 514/563

(57) ABSTRACT

The present invention relates to the use of N-fatty acid-amino acid conjugates to treat, prevent, or manage tissue inflammation, leukocyte adhesion, or pain. The invention includes pharmaceutical compositions comprising N-fatty acid-amino acid conjugates and methods of administering N-fatty acid-amino acid conjugates and pharmaceutical compositions thereof as therapeutic agents.

### CROSS-REFERENCE TO RELATED APPLICATION

This application claims benefit of priority from U.S. provisional patent application Serial No. 60/300,683, filed on Jun. 25, 2001, which is incorporated herein by reference in its entirety.

### STATEMENT AS TO FEDERALLY SPONSORED RESEARCH

This research was sponsored by National Institute on Drug Abuse grant numbers DA09439 and DA09017.

### FIELD OF THE INVENTION

This invention relates to non-psychoactive derivatives of anandamide and their use as anti-inflammatory, analgesic, or anti-leukocyte adhesion agents.

### BACKGROUND OF THE INVENTION

Many diseases and disorders involve inflammation or pain or both. Examples of diseases and disorders that involve inflammation include inflammatory bowel disease, arthritis, rheumatoid arthritis, osteoarthritis, meningitis, appendicitis, systemic lupus erythematosus, multiple sclerosis, psoriasis, and poison ivy. Leukocytes are thought to be major contributors to the inflammatory response, and their ability in this regard is reflected by their adhesiveness to a variety of substrates (Burstein, J. Medicinal Chem., 35(17):3135-3136, 1992). Pain is also a common symptom of many diseases, disorders, and physical conditions. Pain often accompanies inflammation. Any new means of treating, preventing, or managing inflammation, leukocyte adhesion, or pain is desirable.

The most studied pathway for the metabolism of anandamide involves the hydrolysis of the amide bond (Deutsch and Chin, Biochem. Pharmacol., 46:791-796, 1993). This process has been postulated to be a possible mechanism for the physiological regulation of anandamide levels. Products are also produced through the actions of various lipoxygenases (Ueda et al., Biochem. Biophys. Acta, 1254:127-34,1995; Hampson et al., Biochem. Biophys. Acta, 1259:173-9, 1995; Edgemond et al., Mol. Pharmacol., 54:180-8, 1998), and anandamide is a good substrate for cyclooxygenase-2 (COX-2), giving rise to ethanolamide conjugates of Prostaglandin E2 (PGE2) (Yu et al., J. Biol. Chem., 272:21 181-6, 1997). N-fatty acid-amino acid conjugates are analogs of anandamide that consist of amino acid conjugates of several long-chain fatty acids.

### SUMMARY OF THE INVENTION

The invention is based on the discovery that N-fatty acid-amino acid conjugates can be used to treat inflammation, pain, or leukocyte adhesion.

In general, the invention features a pharmaceutical composition that includes a carrier, e.g., a carrier for oral or topical administration (e.g., DMSO), and a therapeutically effective amount of an N-fatty acid-amino acid conjugate, in which the N-fatty acid-amino acid conjugate has the following general formula:

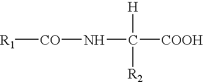

In this general formula R₁ and R₂ represent a variety of chemical group. R₁ can be any N-fatty acid such as any one of myristic acid, palmitic acid, stearic acid, oleic acid, linoleic acid, linolenic acid, eicosatrienoic acid, arachidonic acid, eicosapentenoic acid, or docosatetraenoic acid. R₂ can be any amino acid, such as any one of glycine, D-alanine, L-alanine, D-valine, L-valine, D-leucine, L-leucine, D-isoleucine, L-isoleucine, D-phenylalanine, L-phenylalanine, D-asparagine, L-asparagine, D-glutamine, L-glutamine, or γ-aminobutyric acid.

The invention also features a method of treating inflammation of bodily tissue of a subject (e.g., a human or other mammal, such as a dog, cat, cow, horse, pig, goat, or sheep, or a bird, such as a chicken, duck, or goose) by administering (e.g., orally (e.g., in the form of a tablet or gelatin capsule), topically (e.g., mixed with DMSO), or subcutaneously) to the subject an anti-inflammatory amount (e.g., a dosage of about 0.1-10 or 1.4-2.1 mg/kg of body weight per day, or about 10-700 or 100-150 mg per day) of an N-fatty acid-amino acid conjugate (e.g., N-arachidonylglycine) as described herein.

The invention also features a method of treating pain in a subject by administering (e.g., orally (e.g., in the form of a tablet or gelatin capsule), topically (e.g., mixed with DMSO), or subcutaneously) to the subject an analgesic amount (e.g., a dosage of about 0.1-10 or 1.4-2.1 mg/kg of body weight per day, or about 10-700 or 100-150 mg per day) of an N-fatty acid-amino acid conjugate having the general formula:

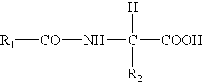

in which R₁ can be any one of myristic acid, palmitic acid, stearic acid, oleic acid, linoleic acid, linolenic acid, eicosatrienoic acid, arachidonic acid, eicosapentenoic acid, or docosatetraenoic acid; and R₂ can be any one of D-alanine, L-alanine, D-valine, L-valine, D-leucine, L-leucine, D-isoleucine, L-isoleucine, D-phenylalanine, L-phenylalanine, D-asp aragine, L-asparagine, D-glutamine, L-glutamine, or γ-aminobutyric acid.

In addition, the invention encompasses a method of reducing leukocyte adhesion in a subject by administering (e.g., orally (e.g., in the form of a tablet or gelatin capsule), topically (e.g., mixed with DMSO), or subcutaneously) to the subject an anti-leukocyte adhesionary amount (e.g., a dosage of about 0.1-10 or 1.4-2.1 mg/kg of body weight per day, or about 10-700 or 100-150 mg per day) of an N-fatty acid-amino acid conjugate (e.g., N-arachidonylglycine) having the general formula described above.

“Bioavailable” refers to the ability of a drug or other substance to be absorbed and used by the body. Orally bioavailable means that a drug or other substance that is taken by mouth can be absorbed and used by the body.

An “effective amount” is an amount of the pharmaceutical composition used in the invention that provides a therapeutic benefit in the treatment, prevention, or management of a condition, disorder, or disease.

An “anti-inflammatory amount” is an amount of the pharmaceutical composition used in the invention that provides a therapeutic benefit in the treatment, prevention, or management of tissue inflammation.

An “analgesic amount,” is an amount of the pharmaceutical composition used in the invention that provides a therapeutic benefit in the treatment, prevention, or management pain.

An “anti-leukocyte adhesion amount” is an amount of the pharmaceutical composition used in the invention that provides a therapeutic benefit in the treatment, prevention, or management of leukocyte adhesion.

A “unit dose” is a single dose, although a unit dose may be divided, if desired.

The term “pharmaceutically acceptable salt” refers to a salt prepared from pharmaceutically acceptable non-toxic acids or bases including inorganic or organic acids. Examples of such inorganic acids are hydrochloric, hydrobromic, hydroiodic, sulfuric, and phosphoric. Appropriate organic acids may be selected, for example, from aliphatic, aromatic, carboxylic and sulfonic classes of organic acids, examples of which are formic, acetic, propionic, succinic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic. Examples of such inorganic bases, for potential salt formation with the sulfate or phosphate compounds of the invention, include metallic salts made from aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylglucamine), and procaine.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The invention offers numerous advantages. N-fatty acid-amino acid conjugates can be used to treat inflammation, pain, and leukocyte adhesion. Unlike many therapeutic compositions, the compositions of the invention are non-psychotropic. In addition, the invention includes a diversity of N-fatty acid-amino acid conjugates. This provides a selection of particular N-fatty acid-amino acid conjugates from which can be chosen those that are most efficacious in treating specific disorders or patients. Furthermore, N-fatty acid-amino acid conjugates are highly bioavailable, so they can be administered orally, topically, subcutaneously, or by various other means. In addition, the N-fatty acid-amino acid conjugates are therapeutically effective without toxic side effects.

Other features and advantages of the invention will be apparent from the following detailed description and from the claims.

### BRIEF DESCRIPTION OF THE DRAWINGS

FIG. 1 is a schematic diagram of the chemical structure of anandamide and its endogenous analog, N-arachidonylglycine (NAGly), as well as of the general chemical formula of the compounds used in the invention and various R₁ and R₂ groups comprised by the general chemical formula.

FIG. 2 is a bar graph that depicts the inhibition of arachidonic acid-induced paw edema in mice. Data were analysed by ANOVA for significance, with 95% significance of vehicle vs. LPS treated cells by ANOVA (P=0.006; N=5) depicted in the graph by “*”.

FIGS. 3A and 3B are graphs that depict the effects of NAGly and anandamide on IL-1β release from human peripheral blood monocytes. Cells were then not stimulated (FIG. 3A) or stimulated (FIG. 3B) with LPS. “*” indicates 95% significance of vehicle vs. LPS treated cells by ANOVA.

FIG. 4 is a graph that depicts the effects of anandamide vs. NAGly on lymphocyte proliferation. The values shown (see “PROLIFERATION INDEX” on the y-axis) are the ratios of the optical density readings from treated and control cells. “*” indicates 95% significance of vehicle vs. NAGly treated cells by ANOVA.

FIG. 5 is a bar graph that depicts the inhibition of proliferation of murine macrophage-derived RAW cells. “*” indicates 95% significance of NAGly treated vs. DMSO-treated cells by ANOVA.

FIG. 6 is a graph that depicts the stimulation of arachidonic acid release in RAW cells.

FIGS. 7A-C depict the inhibition of nuclear factor-kB (NF-kB) activation in human peripheral blood monocytes. FIG. 7A depicts autoradiographs of Donors A and B. FIGS. 7B and 7C are bar graphs that depict the densitometer readings for the gel regions depicted in FIG. 7A.

FIGS. 8A and 8B are bar graphs that depict the effect of NAGly on anandamide levels in intact cultured RAW cells.

FIG. 9 is a bar graph that depicts the effect of orally administered NAGly on blood levels of anandamide in rats.

### DETAILED DESCRIPTION OF THE INVENTION

N-fatty acid-amino acid conjugates, such as N-arachidonylglycine (NAGly), are structurally akin to known lipoamino acids, a class of compounds found in bacteria (Kawazoe et al., J. Bacteriol., 173:5470-5475, 1991; Lerouge et al., Chem. Phys. Lipids, 49:161-166, 1988; Kawai et al., Eur. J. Biochem., 171:73-80, 1988). NAGly was first synthesized (Burstein et al., Proc. ICRS, 131, 1997; Sheskin et al., Advance ACS Abstracts, 1997a; Sheskin et al., J. Med. Chem., 40:659-667, 1997b) as a structural analog of the endogenous cannabinoid anandamide (Devane et al., Science, 258:1946-1949, 1992) and found to lack affinity for cannabinoid CB1 receptors.

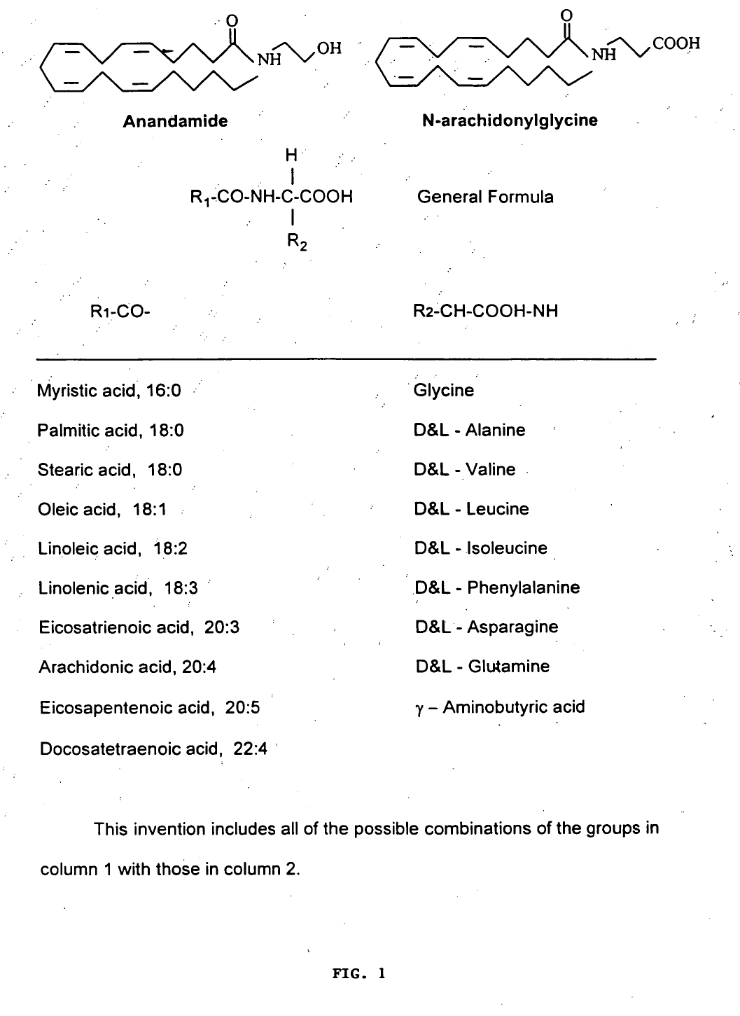

The N-fatty acid-amino acid conjugates (e.g., NAGly) are members of a family of naturally occurring long chain acyl amino acid conjugates. Some, but not all, other members of this family are shown in FIG. 1. NAGly and its N-fatty acid-amino acid conjugate relatives represent the first examples of endogenous regulators of fatty acid amide hydrolase (FAAH) activity, a process that controls tissue levels of the endocannabinoid anandamide.

Methods of Making N-Fatty Acid-Amino Acid Coniugates

In the protocols that follow, R₁ refers to any one of the compounds listed below “R₁—CO—” in FIG. 1, and R₂ refers to any one of the compounds listed below “R₂—CH—COOH—NH” in FIG. 1. Reaction conditions and concentrations of reagents vary depending on the requirements of the R₁ and R₂ groups used. Example 1 describes the preparation of N-arachidonyl glycine (NAGly).

Various protocols can be used to prepare N-fatty acid-amino acid conjugate methyl esters. In general, one prepares a solution of R₂ methyl ester HCl (Aldrich Chemicals) (e.g., 10-100 mequivalents) in a solvent such as methylene chloride (e.g., 6-60 ml) containing a small amount of triethylamine (or other organic soluble bases, such as puridine or morpholine) (e.g., 0.8-8.0 ml) and cool the R₂ ester solution, e.g., to 0-4° C. To the first solution add a second solution of the same amount of R₁—CO— chloride (Nu Chek Inc.) (e.g., 3-30 ml) in methylene chloride and allow to react for a time sufficient to complete the conjugation, e.g., for 60, 120, 180, or 240 minutes, at 0-4° C. Add an equal volume of water to terminate the reaction and extract, e.g., with 50-500 ml of ethyl acetate. Dry the organic layer, e.g., with sodium sulfate, filter, and evaporate to dryness, e.g., under vacuum. The product can be purified using any of a variety of techniques, such as by silica gel column chromatography, and can be eluted with a solvent, such as 1.5% methanol in methylene chloride. Other protocols can also be used.

The following general protocol describes a method to prepare an N-fatty acid-amino acid conjugate. A solution of an N-fatty acid-amino acid conjugate methyl ester (e.g., 0.72-7.2 mmol) in a solvent, such as tetrahyrofuran (e.g., 2.4-24 ml), is treated with a base, e.g., 1 M aqueous lithium hydroxide (e.g., 0.79-7.9 ml). The mixture is stirred (e.g., for 45 minutes) under nitrogen at room temperature followed by evaporation under vacuum. The residue is diluted with water (e.g., 15-150 ml), acidified to a pH of 2-6, e.g., a pH of 3.0, e.g., with 2 N HCl, and extracted, e.g., with ethyl acetate (e.g., 3×20-200 ml). The combined extracts are washed with water, dried with sodium sulfate, and evaporated under vacuum. The product can be purified, e.g., using silica gel column chromatography, and eluted with a solvent, such as 3.5% methanol in methylene chloride. It is then crystallized from acetonitrile/water (m.p. 32-33° C.). Other protocols can be also used.

The general procedures outlined above can be used to prepare the glycine, D&L-alanine, D&L-valine, D&L-leucine, D&L-isoleucine, D&L-phenylalanine, D&L-asparagine, D&L-glutamine, and γ-aminobutyric acid derivatives of several long-chain fatty acids in addition to arachidonic acid (see FIG. 1). These long-chain fatty acids include myristic, palmitic, stearic, oleic, linoleic, linolenic, eicosatrienoic, eicosapentenoic, and docosatetraenoic acids (see FIG. 1).

Inflammation

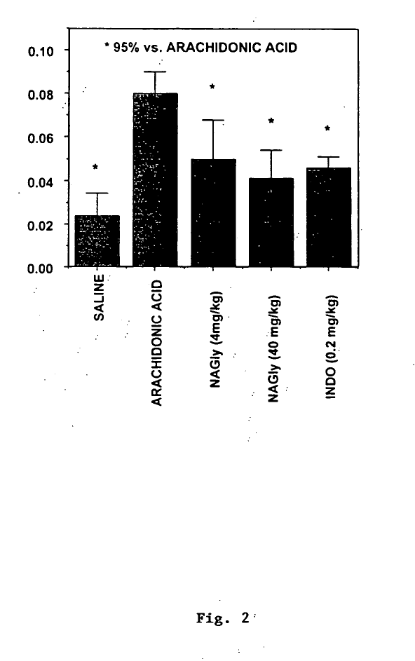

NAGly was tested for anti-inflammatory activity in the rat paw edema model, an assay that has been used previously to detect potential anti-inflammatory agents (Calhoun et al., Agents Actions, 21(3-4):306-9, 1987). Orally administered NAGly (4 mg/kg) reduced paw volumes by one half when compared to vehicle treated rats (see FIG. 2). Endocannabinoids, such as anandamide, generally do not exhibit good bioavailability when given by this route. However, this is not the case for NAGly. In addition, NAGly is only slowly hydrolyzed by FAAH and possesses greater stability than anandamide in vivo. Thus, NAGly is effective in reducing arachidonate-induced paw edema and possesses similar efficacy as an anti-inflammatory agent. Moreover, the data of this invention demonstrate that endogenous NAGly can provide protection against inflammatory reactions.

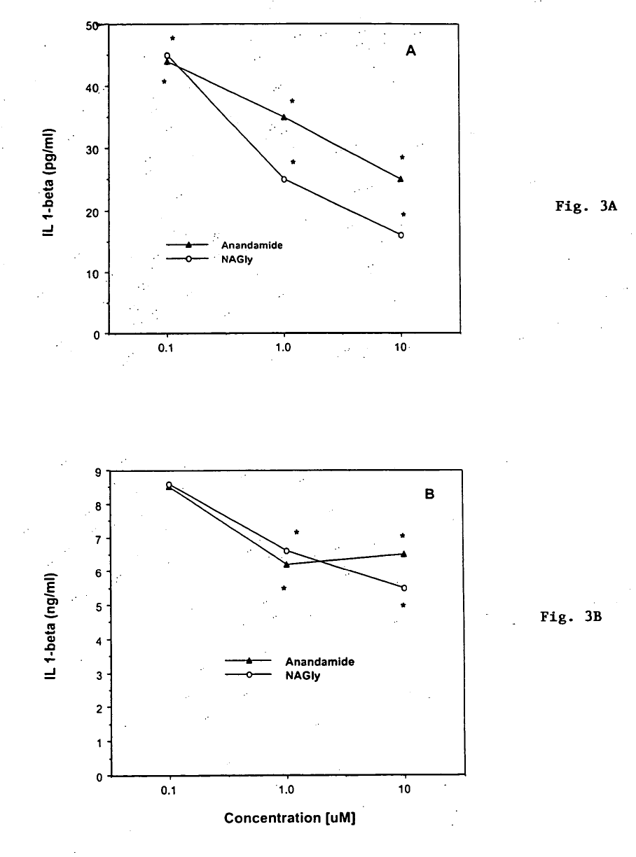

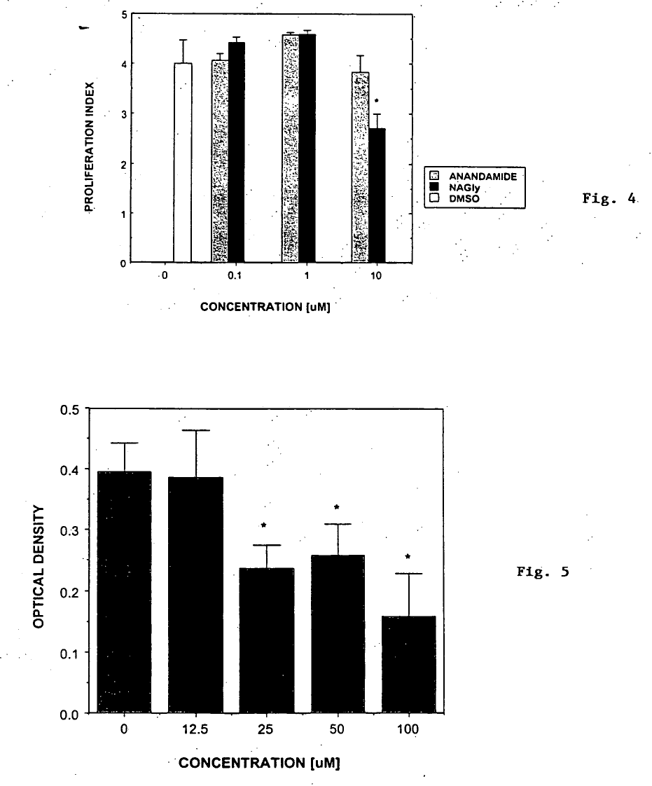

One important site for the anti-inflammatory action of NAGly is the T-cell. A comparison of the effects of anandamide vs. NAGly on T-cell secretion of IL-1β (see FIG. 3) and proliferation (see FIG. 4) revealed a divergence of effects for the two agents. Whereas NAGly was inhibitory in a dose-related fashion over the range of 0.1-10 mM, anandamide had a smaller effect on IL-1β secretion and little or no effect on the proliferation of T-cells derived from human donors. T lymphocyte activation and proliferation are central to the propagation of joint tissue injury in patients with rheumatoid arthritis (Panayi et al., Arthritis Rheum., 35(7):729-35, 1992) and IL-1β optimal proliferation of T-cells. In turn, T cells can influence monocyte activation (Espinoza-Delgado et al., J. Leukoc. Biol., 57(1):13-9, 1995). The high sensitivity of unstimulated cells to suppression of IL-1β (see FIG. 3A) is important therapeutically because the spontaneous release of IL-1β from monocytes of patients with rheumatoid arthritis is usually increased (Goto et al., Ann Rheum Dis, 49(3):172-6, 1990).

The anti-proliferative effect of NAGly was also examined in an established cell line, namely, the RAW rat macrophage-derived cell. Here too, a significant inhibition of cell proliferation was seen, the potency of the effect was lower than that found with the T-cell model (see FIG. 5).

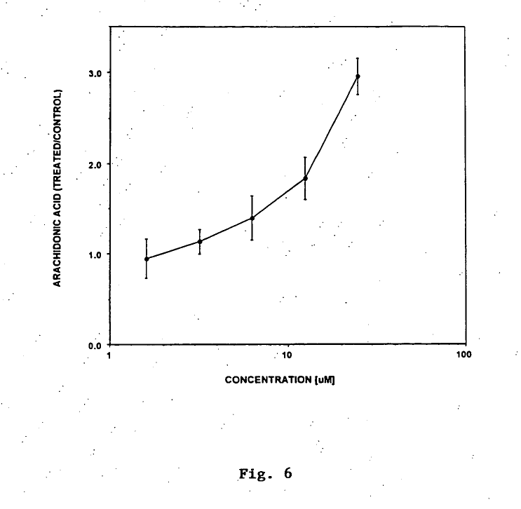

Over the range of 2-32 mM, NAGly caused a three fold stimulation of arachidonic acid release (see FIG. 6). The NAGly could not have been the source of the free arachidonate since it was not radiolabelled, whereas the released fatty acid was radiolabelled.

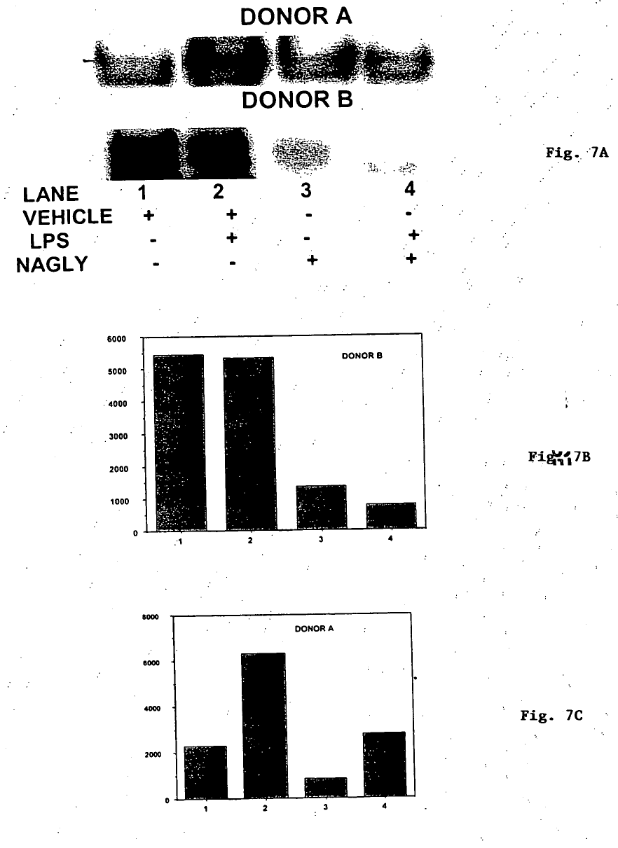

The strong inhibitory effect of NAGly on the activation of the transcription factor NFk-B (see FIGS. 7A-C) suggests a possible mechanism for its anti-inflammatory and antiproliferative activity. A possible mechanism is the activation of the arachidonic acid cascade that could elevate cellular concentrations of inhibitors of NFk-B activation such as the cyclopentenone prostaglandins.

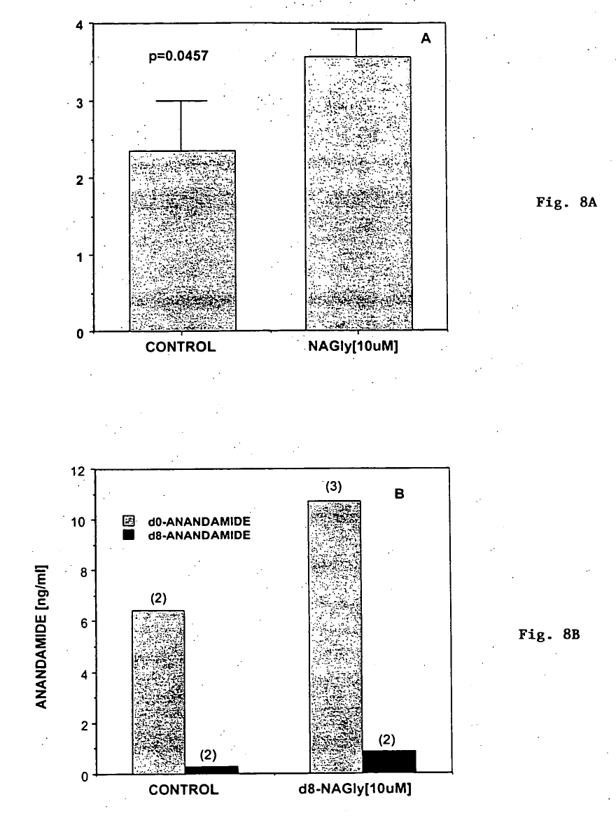

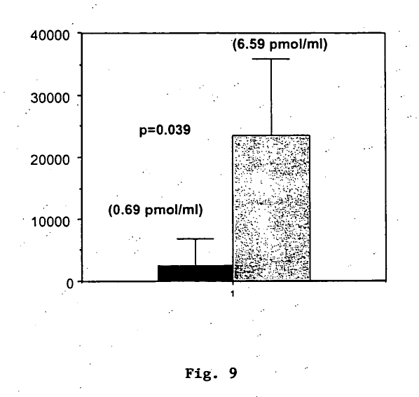

The data that show an increase in anandamide levels in an intact RAW cell culture model following NAGly exposure (see FIGS. 8A and B) support the hypothesis that NAGly may function as an endogenous regulator of FAAH activity and, thereby, of anandamide as well. The possibility that the increased anandamide might be due to arachidonic acid resulting from the breakdown of NAGly is precluded by the lack of labeled anandamide found when deuterated NAGly was used as agonist (see FIG. 8B). In vivo pharmacological support for the hypothesis was obtained by demonstrating increased circulating blood levels of anandamide in rats following the administration of NAGly (see FIG. 9). In any case, these results indicate that NAGly, as well as other N-fatty acid-amino acid conjugates, can be employed as pharmacological agents to control anandamide levels. Furthermore, they can be employed as novel anti-inflammatory agents to modulate endocannabinoid tissue concentrations.

Methods of Use

The invention provides data to demonstrate that N-fatty acid-amino acid conjugates of the invention are effective in reducing inflammation, pain, or leukocyte adhesion. These conjugates can be described by the following general formula:

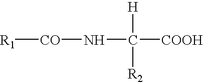

In this general formula R₁ and R₂ represent a variety of chemical groups. R₁ can be any one of myristic acid (16:0), palmitic acid (18:0), stearic acid (18:0), oleic acid (18:1), linoleic acid (18:2), linolenic acid (18:3), eicosatrienoic acid (20:3), arachidonic acid (20:4), eicosapentenoic acid (20:5), or docosatetraenoic acid (22:4). R₂ can be any one of glycine, D-alanine, L-alanine, D-valine, L-valine, D-leucine, L-leucine, D-isoleucine, L-isoleucine, D-phenylalanine, L-phenylalanine, D-asparagine, L-asparagine, D-glutamine, L-glutamine, or γ-aminobutyric acid.

Some N-fatty acid-amino acid conjugates of the invention are particularly effective. For example, any N-fatty acid-amino acid conjugates composed of (1) any one of the R₁ fatty acids listed in FIG. 1 and (2) D-alanine (e.g., N-arachidonyl-D-alanine, N-myristyl-D-alanine), L-alanine (e.g., N-palmityl-L-alanine, N-stearyl-L-alanine), or γ-aminobutyric acid (e.g., N-eicosapentenoyl-γ-aminobutyric acid, N-linolenyl-γ-aminobutyric acid) can be particularly efficacious in treating both inflammation, pain, or leukocyte adhesion.

The N-fatty acid-amino acid conjugates of the invention can be used in both human and veterinary medicine. They can be employed to treat mammals (e.g., humans, mice, rats, dogs, cats, cows, horses, pigs, goats, and sheep), as well as birds (e.g., chickens, ducks, geese), and other animals (e.g., salmon). The actual amounts of N-fatty acid-amino acid conjugates employed in a specific instance will vary, of course, according to the particular species afflicted, the size, age, and condition of the individual, the severity of the inflammation, pain, or leukocyte adhesion to be treated, and the actual method of administration.

Use As Anti-Inflammatory Agents

The conjugates described herein can be used as effective anti-inflammatory agents. For example, N-fatty acid-amino acid conjugates (e.g., NAGly) can be used to effectively treat diseases involving tissue inflammation, especially inflammation associated with long-term illnesses, such as rheumatoid arthritis. Other diseases or disorders that N-fatty acid-amino acid conjugates (e.g., NAGly) can be used to treat include inflammatory bowel disease, arthritis, osteoarthritis, meningitis, appendicitis, systemic lupus erythematosus, multiple sclerosis, poison ivy (and other allergic reactions), and psoriasis. N-fatty acid-amino acid conjugates are bioavailable, so they can be administered orally, topically, subcutaneously, or by various other means. One of ordinary skill in the art would be able to formulate therapeutically effective compositions and dosages of these compounds to treat particular inflammatory diseases.

Defective regulation of T lymphocyte function is observed in diseases such as rheumatoid arthritis (RA) and systematic lupus erythematosus that are characterized by inflammation and tissue injury. N-fatty acid-amino acid conjugates (e.g., NAGly) can be administered to patients who suffer from these diseases to reduce or modulate the level of inflammation. N-fatty acid-amino acid conjugates (e.g., NAGly) have dose-related effects on proliferation of T lymphocytes in vitro. For example, when compared with a control of vehicle (DMSO) treated cells, NAGly is stimulatory at low concentrations and somewhat inhibitory at higher concentrations. Anandamide, on the other hand, shows only inhibition of T-cell proliferation. A clear divergence in the effects of low concentrations of anandamide and NAGly on T-cell proliferation suggests a modulating effect on T cell activation.

The induction of paw edema, in rodents, by the injection of arachidonic acid, can be used as an experimental model for inflammation (Calhoun et al., Agents Actions, 21:306-309, 1987). Administration of non-steroidal, anti-inflammatory drugs prior to induction of paw edema with arachidonic acid leads to a dose-related inhibition of edema (see, e.g., FIG. 2) that are considered predictive of clinical efficacy in humans.

The conditions employed in the paw edema test have been previously described (Calhoun et al., Agents Actions, 21:306-309, 1987; Burstein et al., J. Pharmacology and Experimental Therapeutics, 251:531-535; Burstein et al., J. Med. Chem., 35:3135-41, 1992), with water being substituted for mercury as the displacement medium. Arachidonic acid (1.0 mg) in 25 μl of saline is injected subcutaneously into the plantar surface of the right hindpaw of ether-anesthetized CD-1 female mice (20-25 g) obtained from Charles River Laboratories. The volume of the right foot is then measured to the level of the lateral malleolus by water displacement before treatment and 30 minutes after arachidonic acid injection. An N-fatty acid-amino acid conjugate, in 50 μl of peanut oil, is given p.o. to the mice 30 min prior to the induction of edema by arachidonic acid. The change in paw volume is calculated for each mouse and the significance for each group can be determined by an ANOVA test.

N-fatty acid-amino acid conjugates of the present invention are effective in reducing paw edema at doses ranging from, e.g., 4 to 40 mg/kg by amounts up to more than 50% when compared with untreated control mice that receive only arachidonic acid (see, e.g., FIG. 2). This takes into consideration the increase in paw volume due to the saline vehicle. For example, such doses of NAGly have been shown to give an equivalent response to that shown by 0.2 mg/kg of indomethacin, a well-known nonsteroidal anitinflammatory drug (NSAID) (see FIG. 2). These results clearly demonstrate that N-fatty acid-amino acid conjugates can be effective in reducing arachidonate induced paw edema and thus have efficacy as an anti-inflammatory agents in treating inflammation in humans and other subjects.

Although a variety of mediators contribute to inflammatory responses, it seems clear that the actions of interleukin-1β (IL-1β) and tumor necrosis factor (TNF) are central to progression of joint tissue injury in RA patients. Both cytokines have become targets of therapy for patients with RA. Results of an experiment indicate that both anandamide and NAGly modestly suppress IL-1β secretion from stimulated human peripheral blood mononuclear cells (PBMC) at concentrations of 1 μm and 10 μm (Burstein et al., Prostaglandins & other Lipid Mediators, 61:29-41, 2000). Both compounds act on monocytes directly because NAGly increases activation of isolated lymphocytes. The observations that NAGly, in particular, markedly reduces IL-1β secretion from unstimulated PBMC demonstrates that NAGly serves to modulate spontaneous release from monocytes of IL-1β, which is usually increased in patients with active RA.

Use As Analgesic Agents

The invention also provides the use of certain N-fatty acid-amino acid conjugates (e.g., N-arachidonylleucine, N-stearylphenylalanine, N-palmitylglutamine) as effective analgesic agents. Example 5 illustrates the use of an N-fatty acid-amino acid conjugate, NAGly, to reduce pain in rats in a formalin test. However, those N-fatty acid-amino acid conjugates whose R₂ amino acids are bulkier than glycine are generally more effective for treating pain.

Use As Anti-Leukocyte Adhesion Agents

N-fatty acid-amino acid conjugates (e.g., NAGly) also serve as modulators of leukocyte adhesion. Consequently, the invention also provides the use of certain N-fatty acid-amino acid conjugates (e.g., NAGly) as anti-leukocyte adhesion agents. Example 3, as well as other Examples, demonstrate that N-fatty acid-amino acid conjugates, such as NAGly, can be the used to reduce leukocyte adhesion, for example, in human patients. Also, the method described in Example 3 can be used to test for an N-fatty acid-amino acid conjugate efficacious as an anti-leukocyte adhesion agent.

Pharmacological Formulations

The compositions used in the present invention can be used in both veterinary medicine and human therapy. The prophylactic or therapeutic dose of the composition used in the treatment, prevention, or management of tissue inflammation, acute or chronic pain, or leukocyte adhesion activity will vary with the severity of the condition to be treated and the route of administration. The dose and dose frequency will also vary according to the age, body weight, and response of the individual patient. In general, the total daily dose range of the active ingredient used in this invention would be between about 0.1 and 10 mg/kg of body weight, e.g., between about 0.14 and 7.14 mg/kg of body weight per day; 0.71 and 3.57 mg/kg of body weight per day; or 1.43 and 2.14 mg/kg of body weight per day. For example, these dosages correspond to the following amounts for an average 70 kg adult: between about 7 and 700 mg per day; e.g., 10 and 500 mg per day; 50 and 250 mg per day; or 100 and 150 mg per day. The actual amounts of the active ingredient used in this invention will vary with each case, according to the species of mammal (or other animal), the nature and severity of affliction being treated, and the method of administration. For example, the range of doses for rodents such as mice would be about 1-40 mg/kg of body weight. In general, the compositions used in the present invention are periodically administered to an individual patient as necessary to improve symptoms of the disease being treated. The length of time during which the compositions used in the invention are administered and the total dosage will necessarily vary with each case, according to the nature and severity of the affliction being treated and the physical condition of the subject.

Generally, then, each daily dose is a unit dose, i.e., tablet, cachet or capsule, which contains between about 10 mg to 700 mg of the active ingredient of the invention, e.g., 50 mg to 250 mg, or about 100 mg to 150 mg of the active ingredient (i.e., excluding excipients and carriers). If desired, the daily dose may include two or more unit doses, i.e., tablets, cachets or capsules, to be administered each day.

It may be necessary to use dosages outside these ranges in some cases, as will be apparent to those of ordinary skill in the art. Further, it is noted that the clinician or treating physician will know how and when to interrupt, adjust, or terminate therapy in conjunction with individual patient response.

Any suitable route of administration may be employed for providing the patient with an effective dosage of the composition according to the methods of the present invention. For example, because of their oral bioavailability, the compositions used in the invention may be administered orally. Other suitable routes include, for example, rectal, parenteral (e.g., in saline solution), intravenous, topical, transdermal, subcutaneous, intramuscular, by inhalation, and like forms of administration may be employed. Suitable dosage forms include tablets, troches, dispersions, suspensions, solutions, capsules, patches, suppositories, and the like.

The pharmaceutical compositions used in the methods of the present invention include the active ingredients described above, and may also contain pharmaceutically acceptable carriers, excipients and the like, and optionally, other therapeutic ingredients. In one embodiment, for example, the drug is dissolved in a vegetable oil, such as olive oil or peanut oil, and, optionally, encapsulated in a gelatin capsule.

The compositions for use in the methods of the present invention include compositions such as suspensions, solutions and elixirs; aerosols; or carriers such as starches, sugars, microcrystalline cellulose, diluents, granulating agents, lubricants, binders, disintegrating agents, and the like, in the case of oral solid preparations (such as powders, capsules, and tablets).

Because of their ease of administration, tablets and capsules represent the most advantageous oral dosage unit form, in which case solid pharmaceutical carriers are employed. If desired, tablets may be coated by standard aqueous or nonaqueous techniques.

In addition to the common dosage forms set out above, the compound for use in the methods of the present invention may also be administered by controlled release means and/or delivery devices such as those described in U.S. Pat. Nos. 3,845,770; 3,916,899; 3,536,809; 3,598,123; and 4,008,719, the disclosures of which are hereby incorporated by reference in their entirety.

Pharmaceutical compositions for use in the methods of the present invention suitable for oral administration may be presented as discrete units such as capsules, cachets, or tablets, or aerosol sprays, each containing a predetermined amount of the active ingredient, as a powder or granules, as creams, pastes, gels, or ointments, or as a solution or a suspension in an aqueous liquid, a non-aqueous liquid, an oil-in-water emulsion, or a water-in-oil liquid emulsion. Such compositions may be prepared by any of the methods of pharmacy, but all methods include the step of bringing into association the carrier with the active ingredient that constitutes one or more necessary ingredients. In general, the compositions are prepared by uniformly and intimately admixing the active ingredient with liquid carriers or finely divided solid carriers or both, and then, if necessary, shaping the product into the desired presentation.

For example, a tablet can be prepared by compression or molding, optionally, with one or more accessory ingredients. Compressed tablets can be prepared by compressing in a suitable machine the active ingredient in a free-flowing form, such as powder or granules, optionally mixed with a binder (e.g., carboxymethylcellulose, gum arabic, gelatin), filler (e.g., lactose), adjuvant, flavoring agent, coloring agent, lubricant, inert diluent, coating material (e.g., wax or plasticizer), and a surface active or dispersing agent. Molded tablets can be made by molding, in a suitable machine, a mixture of the powdered compound moistened with an inert liquid diluent. Those skilled in the art will know, or will be able to ascertain with no more than routine experimentation, appropriate pharmacological carriers for said pharmaceutical compositions.

As an another example, the compositions of the invention can be administered through the skin directly to a region of tissue to be treated, e.g., for pain, by use of skin permeation enhancers, such as dimethyl sulphonide (DMSO). An effective dose of an N-fatty acid-amino acid conjugate (e.g., NAGly) can be mixed together with DMSO and then topically applied to the skin of a patient. The mixture can take the form of, for example, a cream, gel, or ointment.

The invention will be further described in the following examples, which do not limit the scope of the invention described in the claims.

### EXAMPLES

Note on Statistical Analysis and Materials

Values are the means±SE for 4 replicates unless otherwise indicated. A one-factor ANOVA evaluation was used followed by a Fisher PLSD post-hoc test to compare the different sets of experimental data for statistical significance. The sets were considered to be meaningfully different at P<0.05.

RAW 264.7 murine monocyte cells were prepared from stock. MEM (minimal Eagle's medium) was purchased from ICN (Costa Mesa, Calif.). Fetal bovine serum and penicillin-streptomycin solution were obtained from GIBCO BRL (Grand Island, N.Y.). d8-arachidonic acid was obtained from Cayman Chemical (Ann Arbor, Mich.). Bovine serum albumin; Sep-Pak Plus C18 cartridges were purchased from Waters Corp. (Milford, Mass.). Thin layer chromatography plates were obtained from EM Science (Gibbstown, N.J.).

### Example 1

### Preparation of N-Arachidonyl Glycine (NAGIy)

N-arachidonyl glycine methyl ester can be prepared as follows. Prepare a solution of 10 mequivalents of glycine methyl ester HCl (Aldrich Chemicals) in 6 ml methylene chloride containing 0.8 ml of triethylamine and cool to 0° C. Add a solution of 10 mequivalents of arachidonyl chloride (Nu Chek Inc.) in 3 ml methylene chloride to the glycine ester solution and allow to react for 180 minutes at 0° C. Add an equal volume of water to terminate the reaction and extract with 50 ml of ethyl acetate. Dry the organic layer with sodium sulfate, filter and evaporate to dryness under vacuum. The product is purified by silica gel column chromatography and is eluted with 1.5% methanol in methylene chloride. The yield is 86%. In addition, HRMS (EI) is m/z 375.2768 (C₂₃H₃₇NO₃ requires 375.2773).

N-arachidonyl glycine can be prepared as follows. A solution of NAGly methyl ester (250 mg, 0.72 mmol) in tetrahyrofuran (2.4 ml) was treated with 1 M aqueous lithium hydroxide (0.79 ml). The mixture is stirred for 45 minutes under nitrogen at room temperature followed by evaporation under vacuum. The residue is diluted with 15 ml of water, acidified pH 3.0 with 2 N HCl and extracted with ethyl acetate (3×20 ml). The combined extracts were washed with water (2×20 ml), dried with sodium sulfate and evaporated under vacuum. The product was purified using silica gel column chromatography and eluted with 3.5% methanol in methylene chloride. It was then crystallized from acetonitrile/water; m.p. 32-33° C. The yield is 86%. In addition information, HRMS (EI) is m/z 361.2641 (C₂₂H₃₅NO₃ requires 361.2617).

### Example 2

### Paw Edema Test for Inflammation

The induction of paw edema, in rodents, by the injection of arachidonic acid, has been used as an experimental model for inflammation (See, e.g., Calhoun et al., Agents Actions, 21:306-309, 1987). Administration of non-steroidal anti-inflammatory drugs (NSAIDs) prior to induction of paw edema with arachidonic acid, leads to a dose-related inhibition of edema that may be considered predictive of clinical efficacy (see FIG. 2).

The conditions were previously described by Calhoun et al. (Agents Actions, 21:306-309, 1987), and by Burstein et al. (J. Pharmacology and Experimental Therapeutics, 251:531-535; J. Med. Chem., 35:3135-41, 1992), with water being substituted for mercury as the displacement medium. Arachidonic acid (1.0 mg in 25 μl of saline) was injected subcutaneously into the plantar surface of the right hindpaw of ether-anesthetized CD-1 female mice (20-25 g) obtained from Charles River Laboratories. The volume of the right foot was measured to the level of the lateral malleous by water displacement before treatment and 30 minutes after arachidonic acid injection. NAGly or analogs, in 50 μl of peanut oil, was given p.o. to the mice 30 minutes prior to the induction of edema by arachidonic acid. The change in paw volume was calculated for each mouse and the significance for each group was determined by an ANOVA test.

NAGly of the present invention is effective in reducing paw edema at doses of 4 and 40 mg/kg by more than 50% when compared with untreated control mice that received only arachidonic acid (see FIG. 2). This takes into consideration the increase in paw volume due to the saline vehicle. These doses of NAGly gave an equivalent response to that shown by 0.2 mg/kg of indomethacin, a well known NSAID. These results clearly demonstrate that NAGly is effective in reducing arachidonate induced paw edema and has efficacy as an anti-inflammatory agent.

### Example 3

### Leukocyte Adhesion Test

Leukocytes are thought to be major contributors to the inflammatory response, and their ability, in this regard, is reflected by their adhesion to a variety of substrates. Following the procedure of Audette and Burstein (Life Sci., 47:753-759, 1983), peritoneal cells from female CD-1 mice (20-25 g) are collected at ninety (90) minutes following oral administration of the test compound or vehicle (50 μL of peanut oil). Cells from each treatment group (N=3) are pooled, and equal numbers of cells are aliquoted into six culture dish wells (1.9 cm² area). After incubation for 18-20 hours, nonadhering cells are removed and the remaining cell monolayer quantitated by DNA measurement. Cell viability is monitored by Trypan Blue exclusion.

### Example 4

### Test for Antinociception

One can measure the analgesic activity of pharmacologic agents based on the reaction time of mice to lick their forepaws and/or jump after being placed on an aluminum plate heated to, and maintained at, 54-56° C. (Kitchen and Green, Life Sci., 33:669-672, 1983)).

An aluminum surface is maintained at 55±1° C. by circulating water through passages in the metal. A clear plastic cylinder, 18 cm in diameter and 26 cm high, is placed on the surface to prevent escape. The end point is taken when the mouse either performed a hind paw lick or jumped off the surface; in no case are the animals kept more than 30 seconds on the plate. Mice are never used more than one time; control values are measured at 11 a.m. and test values at 2 p.m. The compounds to be tested are administered orally ninety (90) minutes before the hot plate test. The percent change in response time (latency) is calculated by comparing the mean of the control values with the mean of the test values and statistical significance determined by a paired t-test.

### Example 5

### Formalin Test for Pain Reduction

Rats received intraplantar injections of formalin in the hindpaw, and pain behavior consisting of lifting and licking of the injected paw was observed. Drug or co-vehicle was coinjected with the formalin solution (4.5% formalin in 10% DMSO, 100 μl, s.c., ipl.). Formalin injection elicited a robust two-phase pain response, consisting of a brief first phase (acute pain), a transient remittance of pain behavior, and a prolonged second phase (tonic pain). Neither glycine nor arachidonic acid (275 nmol) suppressed pain when coinjected with formalin and NAGly had no effect on the first phase of the formalin response. However, in the second phase, 275 mmol NAGly markedly suppressed the pain response elicited by formalin. No behavioral abnormality was observed in the drug-treated animals.

A combination of mechanisms appears to contribute to phase 2 pain behavior in the formalin test. Both persistent peripheral nociceptor discharge and the ensuing central sensitization in the spinal cord are important in the initiation and maintenance of the spontaneous pain, allodynia, and hyperalgesia (Puig and Sorkin, Pain, 64:345-355, 1996; Taylor et al., J. Neurosci., 15:7575-7584, 1995; Dickenson and Sullivan, Pain, 30:349-360, 1987; Dickenson and Sullivan, Neurosci. Lett., 83:207-211, 1987). This tonic pain phase has been likened to persistent postoperative pain (reviewed in Taylor et al., J. Neurosci., 15:7575-7584, 1995; Abram, Anesthesiology, 86:1015-1017, 1997). The efficacy of peripherally administered NAGly in inhibiting phase 2 pain behavior suggests that NAGly likely suppressed the formalin-induced hyperactivity in nociceptive afferents either directly on the nerve, or indirectly by modulating their immediate interstitial environment. Since either action would minimize central sensitization leading to reduced pain following tissue injury, the suppression of formalin-induced pain by NAGly has relevance to postoperative and chronic pain states.

### Example 6

### Measurement of Cataleptic Effects

The cataleptic response in mice or other laboratory animals is measured using the ring test described by Pertwee (Br. J. Pharmacol., 46:753-763, 1972). Mice are placed on a horizontal wire ring 5.5 cm in diameter, which is attached to a 16 cm vertical rod. The hind paws and fore paws are placed at opposite sides of the ring. It is important that the ambient temperature be maintained at 30° C., and that the environment be free of auditory stimuli and bright lights. The response is calculated as the fraction of time the mouse is immobile over a 5 minute test period. Measurements are done between a fixed time, e.g., 2 p.m. to 4 p.m.

### Example 7

### Preparation of Capsules

A large number of unit capsules are prepared by filling standard two-piece hard gelatin capsules each with the desired amount of powdered active ingredient as described above, 150 mg of lactose, 50 milligrams of cellulose, and 6 mg of magnesium stearate.

### Example 8

### Preparation of Soft Gelatin Capsules

A mixture of active ingredient in a digestible oil such as soybean oil, lecithin, cottonseed oil or olive oil is prepared and injected by means of a positive displacement pump into gelatin to form soft gelatin capsules containing the desired amount of the active ingredient. The capsules are washed and dried for packaging.

### Example 9

### NAGly Modulation of Inflammatory Responses and Cell Proliferation

To illustrate that NAGly plays a role in modulating both inflammatory responses and cell proliferation, NAGly was compared with anandamide for its effect on the release of the inflammation mediator IL-1β from lipopolysaccaride (LPS) stimulated human peripheral blood monocytes (PBM). Radiolabelling was carried out according to the method of Example 10.

NAGly was found to be a more effective inhibitor than anandamide in both stimulated and unstimulated cells. In the range of 0.1 to 1.0 mM, NAGly produced an inverse dose-related increase in the proliferation of anti CD3, CD4 treated human T lymphocytes, while anandamide showed either no effect or a modest inhibition of proliferation.

NAGly suppresses the secretion of IL-1β from LPS stimulated peripheral blood mononuclear cells in a concentration related manner over the range of 0.1-10 mM (see FIG. 3B). Perhaps of even more interest is the striking reduction of IL-1β release from PBM stimulated only by virtue of adherence to the culture plates (see FIG. 3A). Cells (2×10⁶) were incubated for 60 minutes with either agent at the indicated concentrations. Vehicle (0.5% DMSO) treated cells served as controls. Cells were then not stimulated (FIG. 3A) or stimulated (FIG. 3B) with LPS (10 ng/ml) for 18 hours, at which time supernatant IL-1β was measured by ELISA. Values shown are the means obtained from triplicate samples (Control A=826 pg/ml; Control B=8.68±0.5 ng/ml). Anandamide was less effective than NAGly in both models.

In contrast to a prior observation on the effect of NAGly (Burstein et al., Prostaglandins Other Lipid Mediat, 61(1-2):29-41, 2000) on proliferation of CD3-CD4 antibody-stimulated T-cells where a biphasic effect was seen, the effect on PHA stimulated cells was only inhibitory (see FIG. 4). Isolated human T-lymphocytes (5×10⁵) were cultured in 96 well microtiter plates and treated with NAGly or DMSO for 60 minutes. The cells were then stimulated with PHA (10 ng/ml) and incubated for 72 hours. A control set treated with NAGly/DMSO not stimulated with PHA was incubated in parallel. Cell numbers were determined by measuring BrDU incorporation for 24 hours. The values shown (see “PROLIFERATION INDEX” on the y-axis) are the ratios of the optical density readings from treated and control cells. Anandamide showed no significant effects over the 0.1-10 mM concentration range.

FIG. 6 shows the stimulatory effect of non-radiolabelled NAGly on arachidonic acid release in RAW cells whose phospholipid pools contain radiolabelled arachidonate. Radiolabelled arachidonic acid was incorporated into the cellular lipid pools for 20 hours. The cells were then exposed to NAGly/DMSO for 60 minutes and radioactivity in the media was measured. The results are expressed as the ratio of NAGly treated to DMSO treated cells and are the means of three replicates±SD (release from control cells=8,800±1600 dpm/ml). Over the concentration range of 1.6-32 mM, a 300% increase in labeled, free arachidonic acid was found in the media when compared to vehicle treated control values. Media from cells in the control group contained 8,800±1600 dpm.

Note that changes in the regulation of T-cell function are observed in diseases such as rheumatoid arthritis and systemic lupus erythematosis.

### Example 10

### Synthesis of Deuterated N-Arachidonylglycine (d8-NAGly)

To a solution of N-hydroxysuccinimide (4 mg) in 5 ml of ethyl acetate, a solution of 10 mg d8-arachidonic acid (5Z,8Z,11Z,14Z-eicosatetraenoic-5,6,8,9,11,12,14,15-d8 acid) (>98 atom% D) was added followed by 9 mg of dicyclohexylcarbodiimide. The mixture was allowed to react for 24 hours at room temperature at which time 10 mg of glycine in a mixture of dioxane-KOH-NaHCO3 (2 ml) was added and reacted for a further 48 hours at 40° C. The mixture was then acidified with HCl, extracted with ethyl acetate and the product isolated by thin layer chromatography (acetonitrile, 96; water, 4). The identity and deuterium content were confirmed by mass spectral analysis.

### Example 11

### Measurement of Anandamide Levels in Cell Culture and in Blood

In this study, evidence was sought to determine whether NAGly would cause a rise in anandamide levels in a physiological system, namely, an intact cell model such as the cultured macrophage RAW cell line. In addition, experiments were done to rule out the possibility that NAGly might serve as a metabolic precursor for anandamide—an effect that would result in an increase in anandamide concentration.

Anandamide and NAGly levels were measured in plasma extracts by HPLC-MS/MS with d8-anandamide added as an internal standard. Reverse phase HPLC was performed on a 100×1 mm i.d. column packed with ODS Hypersil® (3 μm, 120 A pore size, Keystone Scientific, Inc., Bellefonte, Pa.). A Rheos® 2000 micro HPLC pumping system was used to pump the mobile phase (90% methanol, 10% 0.05% pH 5.7 aqueous ammonium acetate buffer) at 50 μl/minute. The outlet from the column was connected directly to the electrospray ion source of a Finnigan® LCQ quadrupole ion trap mass spectrometry system.

Positive ion electrospray ionization was used with the source at 4500 V, the capillary at 200° and the nitrogen sheath gas at a relative setting of 60. NAGly eluted at 1.4 minutes and was detected by MS2 of its MH+ ion (m/z 362.2) with an isolation window of 2.5 Th and relative collision energy (CID) of 29%. Full product ion spectra were collected from m/z 95-370 and peak areas from ion plots of m/z 287.2 were used for quantitation. Anandamide and d8-anandamide were similarly detected using their MH+ ions (m/z 348.2 and 356.2) as precursors with isolation windows of 2.5 Th and CID at 30%. Full product ion spectra were collected from m/z 200-370 and peak areas from ion plots of m/z 286.2 and 292.2 were used for anandamide and d8-anandamide respectively. Concentrations of NAGly and anandamide were calculated from their peak area ratios to the internal standard with reference to an external calibration curve.

Cells were grown as described previously (Pestonjamasp and Burstein, Biochim Biophys Acta, 1394(2-3):249-60, 1998). Values shown for anandamide were obtained by mass spectrometric analysis. Data in FIG. 8A were obtained using non-deuterated NAGly (10 μM); data in FIG. 8B resulted from treatment of cells with deuterated NAGly (10 mM). Treatment of RAW cells with 10 mM NAGly caused a 50% elevation of basal concentrations of anandamide as determined by mass spectral analysis (see FIG. 8A). To exclude the possibility that NAGly was a precursor for the increased anandamide, the experiment was repeated except that only deuterium labeled NAGly (d8-NAGly) was used in the treatment. The label was contained entirely in the arachidonyl portion of the molecule. FIG. 8B shows the data obtained indicating that virtually all of the 50% increase (˜3 ng/ml) in anandamide consists of unlabelled material. Only an insignificant increase (<0.5 ng/ml) in d8-anandamide was found to occur when the cells were treated with d8-NAGly.

The data presented here, using an intact cell culture model, support the hypothesis that NAGly functions as a pharmacological regulator of in vivo anandamide levels. The possibility that the increased anandamide might be due to arachidonic acid resulting from the breakdown of NAGly is precluded by the lack of labeled anandamide found when deuterated NAGly was used as agonist (FIG. 8B).

### Example 12

### Method for PHA Stimulated T-Cell Proliferation

Cell proliferation in vitro was measured by incorporation of 5-bromo-2′-deoxyuridine (BrDU) instead of thymidine into the DNA of proliferating cells. In these experiments, isolated T-lymphocytes were treated with NAGly or vehicle (DMSO) for 60 minutes in RPMI containing 2% autologous serum. Cells (2×10⁵ per well) were then cultured in 96 well, flat bottomed microtiter plates and stimulated with PHA (10 ng/ml) or not stimulated. Cultures were maintained for 72 hours in a standard cell incubator at 37° C. BrDU was then added to all wells and cells are then reincubated for 24 hours. The medium was removed and the cells were fixed and DNA was denatured. Anti-BrDU-peroxidase was added and the immune complex was detected by substrate (tetramethylbenzidine) reaction. The reaction was stopped with 25 ml sulfuric acid (IN) and the product was quantified by measuring the absorbance at 492 nm with reference at 650 nm. Absorbance correlated directly to the amount of DNA synthesis, and, thereby, to the number of proliferating cells in each culture.

### Example 13

### RAW Cell Proliferation (MTT Assay)

Cells were maintained in MEM containing 0.5% serum overnight. On the day of assay, 2×104 cells/well were placed in 24 well plates containing 1 ml MEM and 10% fetal calf serum for two hours after which NAGly was added in a volume of 10 ml DMSO. After a 24 hour incubation period, 100 ml MTT (Sigma Co) solution (5 mg/ml in PBS) was added to each well and incubated for 2 hours at 37° C. Then, cells were triturated in 1.1 ml isopropanol MTT solubilization solution (Sigma Co). 300 ml of this mixture was then transferred in duplicate to a 96 well plate and the optical density recorded using a ThermoMax® microplate reader at 570/650 nM absorbance. Based on a standard curve with known numbers of cells, experimental readings were in the linear range for cell numbers.

### Example 14

### Arachidonic Acid Release in RAW Cells

FIG. 6 shows the stimulatory effect of non-radiolabelled NAGly on arachidonic acid release in RAW cells whose phospholipid pools contain radiolabelled arachidonate.

The cells were grown in MEM with 1% penicillin-streptomycin and 10% fetal bovine serum at 37° C. under 95% oxygen:5% carbon dioxide to about 85% confluence. Cells were contained in 24-well plates and labeled with 100,000 dpm/ml/well of ³H or ¹⁴C arachidonic acid at 37° C. for 20 hours (Pestonjamasp and Burstein, Biochim Biophys Acta, 1394(2-3):249-60, 1998). The wells were washed 4 times with 0.5% BSA in MEM followed by incubation in 1 ml 0.1% BSA-MEM for 60 minutes. NAGly in DMSO (10 μl) was then added to each well and the incubation continued for the indicated time. Control values were obtained with 10 μl of vehicle. Media were collected, centrifuged at 3000×g to remove cells, and 0.1 ml withdrawn and assayed for radioactivity by liquid scintillation counting.

Over the concentration range of 1.6-32 mM, a 300% increase in labeled, free arachidonic acid was found in the media when compared to vehicle treated control values. Media from cells in the control group contained 8,800±1600 dpm.

### Example 15

### NFk-B Inhibition Studies in PBM

Nuclear factor k-B (NFk-B) is a transcription factor that is believed to mediate inflammatory responses in several pathological conditions (Perkins, Trends Biochem Sci, 25(9):434-40, 2000). Thus, drugs or endogenous agents that inhibit its activation in vitro may also show anti inflammatory action in vivo. Cannabinoid acids such as ajulemic acid are potent inhibitors of this process so that it was of interest to test whether NAGly would also exhibit this type of behavior.

PBM from two donors were prepared and treated separately. Cell extracts were then obtained and analyzed for DNA binding of NF-KB, as determined by autoradiographic-PAGE measurement, according to the following method. Isolated cells (5×10⁶ per sample) were treated with NAGly (10 mM) or DMSO for 1 hour, then stimulated with LPS (10 ng/ml) for 60 minutes. Media were then removed and the cells were transferred to 1.5 ml microcentrifuge tubes and washed 2 times with ice cold PBS. After centrifugation at 10,000 rpm, PBS was removed and 0.5 ml of cytoplasmic extraction buffer (buffer A) was added to the cell pellet. Tubes were vortexed and placed on ice for 10 minutes, then centrifuged at 12,000 rpm for 5 minutes. Cells were washed once with buffer A, which was then added to previous cytosol extract fraction. Cytoplasmic extracts were saved at −80° C. until use. 0.1 ml of cold nuclear extraction buffer (buffer B) was then added to precipitated nuclei and incubated for 60-120 minutes in ice. Nuclear extracts were obtained by centrifugation at 12,000 rpm for 10 minutes and saved at −80° C. until use. (Extraction buffer A is a hypotonic solution and contains HEPES, KCl, EDTA, EGTA, DTT, PMSF, leupeptin, antipain, aprotinin, and NP-40 detergent. Extraction buffer B is a high salt buffer and contains all of the above agents plus glycerol.) The protein concentrations of the nuclear extracts were determined by the Bradford protein assay (Coumassie blue staining). 1-5 g of protein were required for assay. Equal protein aliquots were used from each sample for the NFk-B DNA binding assay. NFk-B consensus oligonucleotide (Pharmacia) was end-labeled with ³²P-ATP utilizing the oligonucleotide 3′ end labeling system (NEN). Excess cold NFk-B was used as a specific competitor; Poly DI-DC was used to differentiate non-specific competitors that may have be present. The DNA binding reaction was performed and samples were run on a 5% polyacrylamide electrophoresis gel (Hoeffer) 200 volts for 2 hours at 4° C. The gel was dried and an autoradiograph was performed by exposing to Kodak Xomat film. Gels were scanned and analysed utilizing the Adobe software for PC.

FIG. 7A depicts autoradiographs of Donors A and B. FIGS. 7B and 7C are bar graphs that depict the densitometer readings for the gel regions depicted in FIG. 7. The data in FIG. 7 show that, in human PBM, treatment with 10 mM NAGly followed by LPS stimulation, effectively reduces the binding of NF k-B to DNA (Lane 4) when compared with stimulated cells that received only LPS (Lane 2). The data in Lane 3 show the effect of NAGly on cells not stimulated with LPS. The unexpectedly high value seen in FIG. 7, Lane 1B, the vehicle treated control from donor B, may be due to the physiological status of the donor such as some type of inflammation. The results were generated using PBM obtained from two different, presumably normal, drug-free donors.

### Example 16

### Inhibition of RAW Cell Proliferation

Exposure of RAW cells to NAGly under the following conditions resulted in a dose-related inhibition of cell proliferation (see FIG. 5): cells were prepared and treated with NAGly/DMSO for 24 hours. Values shown in FIG. 5 are optical density readings obtained using the MTT assay and are directly proportional to the numbers of cells. They are the means of four replicates±SD. Significant decreases in cell numbers were found at 25, 50 and 100 mM concentrations following 24 hours of treatment. The values shown are the optical density readings obtained from the MTT assay where a value of 0.5 is approximately equivalent to 2×10⁴ cells. Vehicle treated cells served as the control and gave a reading of 0.396±0.047 O.D. units. The data shown is representative of three independent experiments.

FIG. 17. In Vivo NAGlY-Induced Elevation of Circulating Levels of Anandamide in Rats

It was demonstrated, in in vivo pharmacological results in rats, that increased levels of anandamide in circulating blood levels followed the oral administration of NAGly (FIG. 4). An extension of cell culture experiments to an in vivo model was carried out to see whether the oral administration of NAGly to rats would result in an increase in blood levels of anandamide. An experiment showed that the administration of 10 mg/kg of NAGly to rats resulted in a rapid rise in blood levels of NAGly (60-80 pmol/ml) demonstrating that a significant fraction of an orally administered dose reaches potential tissue target sites. Using a protocol similar to that reported for a study on the anandamide transport inhibitor AM404 (Burstein et al., J Pharmacol Exp Ther, 251(2):531-5, 1989), experiments using 10 mg/kg of NAGly p.o. were performed. Male Spague-Dawley (150 g, n=3) were given NAGly [10 mg/kg] p.o. and compared with a control group (n=3) given safflower oil. Blood samples were drawn 45 minutes later and their anandamide concentrations determined by mass spectroscopic analysis. The values shown are means of the peak areas±SD of 2-3 determinations per sample. The equivalent concentrations in pmol/ml of blood are shown in brackets. An ANOVA comparison yielded a p value of 0.039. Mass spectroscopic analyses showed a nearly ten-fold rise in blood levels of anandamide at 45 minutes post treatment time when compared with vehicle-treated animals (FIG. 9). The result shown is representative of that obtained in two separate experiments. This, as well as other results disclosed herein, show that NAGly is a good template molecule for the design of pharmacological agents to control anandamide levels.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating inflammation of bodily tissue of a subject, the method comprising administering to the subject an anti-inflammatory amount of an N-fatty acid-amino acid conjugate having the general formula: 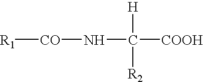
wherein R₁ can be any one of myristic acid, palmitic acid, stearic acid, oleic acid, linoleic acid, linolenic acid, eicosatrienoic acid, arachidonic acid, eicosapentenoic acid, or docosatetraenoic acid; and R₂ can be any one of glycine, D-alanine, L-alanine, D-valine, L-valine, D-leucine, L-leucine, D-isoleucine, L-isoleucine, D-phenylalanine, L-phenylalanine, D-asparagine, L-asparagine, D-glutamine, L-glutamine, or γ-aminobutyric acid.

2. The method of claim 1, wherein the anti-inflammatory amount of the N-fatty acid-amino acid conjugate is administered orally, topically, or subcutaneously.

3. The method of claim 1, wherein the anti-inflammatory amount of the N-fatty acid-amino acid conjugate is administered in the form of a tablet or gelatin capsule.

4. The method of claim 1, wherein the anti-inflammatory amount of the N-fatty acid-amino acid conjugate is administered mixed with DMSO.

5. The method of claim 1, wherein the anti-inflammatory amount of the N-fatty acid-amino acid conjugate is a dosage of about 0.1-10 mg/kg of body weight per day.

6. The method of claim 1, wherein the anti-inflammatory amount of the N-fatty acid-amino acid conjugate is a dosage of about 1.4-2.1 mg/kg of body weight per day.

7. The method of claim 1, wherein the anti-inflammatory amount of the N-fatty acid-amino acid conjugate is a dosage of about 10-700 mg per day.

8. The method of claim 1, wherein the anti-inflammatory amount of the N-fatty acid-amino acid conjugate is a dosage of about 100-150 mg per day.

9. The method of claim 1, wherein the N-fatty acid-amino acid conjugate is N-arachidonylglycine.

10. The method of claim 1, wherein R₂ is D-alanine, L-alanine, or γ-aminobutyric acid.

11. A method of treating pain in a subject, the method comprising administering to the subject an analgesic amount of an N-fatty acid-amino acid conjugate having the general formula: 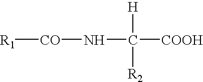
wherein R₁ can be any one of myristic acid, palmitic acid, stearic acid, oleic acid, linoleic acid, linolenic acid, eicosatrienoic acid, arachidonic acid, eicosapentenoic acid, or docosatetraenoic acid; and R₂ can be any one of D-alanine, L-alanine, D-valine, L-valine, D-leucine, L-leucine, D-isoleucine, L-isoleucine, D-phenylalanine, L-phenylalanine, D-asparagine, L-asparagine, D-glutamine, L-glutamine, or γ-aminobutyric acid.

12. The method of claim 11, wherein the analgesic amount of the N-fatty acid-amino acid conjugate is administered orally, topically, or subcutaneously.

13. The method of claim 11, wherein the analgesic amount of the N-fatty acid-amino acid conjugate is administered in the form of a tablet or gelatin capsule.

14. The method of claim 11, wherein the analgesic amount of the N-fatty acid-amino acid conjugate is administered mixed with DMSO.

15. The method of claim 11, wherein the analgesic amount of the N-fatty acid-amino acid conjugate is a dosage of about 0.1-10 mg/kg of body weight per day.

16. The method of claim 11, wherein the analgesic amount of the N-fatty acid-amino acid conjugate is a dosage of about 1.4-2.1 mg/kg of body weight per day.

17. The method of claim 11, wherein the analgesic amount of the N-fatty acid-amino acid conjugate is a dosage of about 10-700 mg per day.

18. The method of claim 11, wherein the analgesic amount of the N-fatty acid-amino acid conjugate is a dosage of about 100-150 mg per day.

19. The method of claim 11, wherein R₂ is D-alanine, L-alanine, or γ-aminobutyric acid.

20. A pharmaceutical composition comprising an N-fatty acid-amino acid conjugate and a carrier for oral or topical administration, wherein said N-fatty acid-amino acid conjugate has the general formula 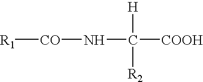
wherein R₁ can be any one of myristic acid, palmitic acid, stearic acid, oleic acid, linoleic acid, linolenic acid, eicosatrienoic acid, arachidonic acid, eicosapentenoic acid, or docosatetraenoic acid; and R₂ can be any one of glycine, D-alanine, L-alanine, D-valine, L-valine, D-leucine, L-leucine, D-isoleucine, L-isoleucine, D-phenylalanine, L-phenylalanine, D-asparagine, L-asparagine, D-glutamine, L-glutamine, or γ-aminobutyric acid.
